(19) United States
(12) Patent Application Publication
(10) Pub. No.: US 2009/0196935 A1
(43) Pub. Date: Aug. 6, 2009

(54) Pharmaceutical Capsules Comprising Extended Release Dipyridamole Pellets

(76) Inventors: Salah U. Ahmed, New City, NY (US); Pruthvipathy R. Katikaneni, Boonton, NJ (US); Yanming Zu, Highland Mills, NY (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/364,241

(22) Filed: Feb. 2, 2009

Related U.S. Application Data
(60) Provisional application No. 61/025,743, filed on Feb. 1, 2008.

(51) Int. Cl. A61K 9/14; A61K 31/519; A61P 25/00

(52) U.S. Cl. 424/494; 424/490; 514/262.1; 424/497

(57) ABSTRACT

The present invention is directed to pharmaceutical capsules comprising extended release formulations of dipyridamole, processes for preparing such dipyridamole extended release formulations and their use in the treatment of stroke.

### CROSS REFERENCE TO RELATED APPLICATIONS

This application claims the benefit of the filing date of U.S. Patent Application No. 61/025,743, filed Feb. 1, 2008, which is incorporated herein by reference in its entirety.

### BACKGROUND OF THE INVENTION

1. Field of Invention

The present invention is directed to pharmaceutical capsules comprising extended release formulations of dipyridamole, processes for preparing such pharmaceutical formulations and their use in the treatment of stroke.

2. Background

Dipyridamole (2,6-bis-(diethanolamino)-4,8-dipiperidino-(5,4-d)-pyrimidine) displays anti-thrombotic and anti-aggregatory activity. Dipyridamole exhibits a relatively short biological half-life of less than one hour. Therefore, extended release formulations of dipyridamole, which provide a continual administration of active ingredient over time, are preferred. Dipyridamole is soluble in acidic mediums with a pH below 4 and is practically insoluble in water. Therefore, dipyridamole is readily absorbed in the more acidic regions of the upper gastrointestinal tract, but remains insoluble in the more basic regions of the intestine.

To obtain a constant level of dipyridamole in the blood, it is advantageous to formulate a dipyridamole dosage form that releases dipyridamole at a controlled rate and at a defined pH. Acidic components can be co-administered with dipyridamole to maintain a defined pH level throughout administration. See U.S. Pat. Nos. 4,361,546, 4,367,217, 4,427,648, and 6,015,577.

However, dipyridamole is chemically incompatible with some acidic components. Acids react with dipyridamole to form hygroscopic salts and dipyridamole-acetic acid esters, thereby causing the dipyridamole to spoil.

Dipyridamole can also be administered with other active ingredients, such as aspirin. Aspirin (acetylsalicylic acid) is an inhibitory substance which counteracts the aggregation of human blood platelets by inhibiting cyclooxygenase and thereby inhibiting the biosynthesis of the aggregation-promoting thromboxane A₂. Dipyridamole and aspirin can have a synergistic effect on thrombocyte aggregation and can be administered in the treatment of stroke, for example, for reducing the risk of stroke in patients who have had transient ischema of the brain or ischemic stroke due to thrombosis.

Dipyridamole and aspirin can be administered as an anti-thrombotic agent which inhibits blood platelet aggregation in humans and animals; the combination prevents the formation and persistence of venous and arterial blood clots and thus prevents temporary ischemic episodes and helps to prevent cardiac infarction and strokes. The combination of dipyridamole and aspirin is also highly suitable for preventing the formation and persistence of clots in the case of arteriosclerosis or after operative intervention and conditions which involve a tendency to thrombosis.

However, aspirin contains traces of acetic acid that are formed by cleavage of the aspirin during storage. The free acetic acid produced by the aspirin can degrade dipyridamole contained in dosage forms that provide both dipyridamole and aspirin in same manner as discussed above.

Thus, co-administration of dipyridamole together with acidic excipients and/or active ingredients requires a formulation that separates dipyridamole from the acidic components and active ingredients, such as aspirin.

### BRIEF SUMMARY OF THE INVENTION

What is needed is a stable dipyridamole composition in which the dipyridamole has a high bioavailability and is separated from any acidic components present in the dosage form.

The present invention is directed to a pharmaceutical capsule comprising a dipyridamole extended release pellet comprising: (i) a core comprising a first organic acid, wherein the core has an aspect ratio of 1.2 or greater; (ii) a first coating layer comprising a second organic acid and a binder, wherein the first coating layer encompasses the core; (iii) a second coating layer comprising a binder, wherein the second coating layer encompasses the first coating layer, and wherein the second coating layer is substantially free of an organic acid; (iv) a drug layer comprising dipyridamole and a binder, wherein the drug layer encompasses the second coating layer; and (v) an extended release layer comprising an enteric polymer, wherein the extended release layer encompasses the drug layer; wherein the dipyridamole is not in contact with the first organic acid and the second organic acid.

In some embodiments, the composition of the core and the first coating layer encompassing the core has an aspect ratio of about 2 to about 1. In some embodiments, the ratio of the first organic acid and second organic acid to the dipyridamole is about 0.1 to about 2, by weight. In some embodiments, the first organic acid and the second organic acid are independently selected from: tartaric acid, citric acid, fumaric acid, succinic acid, malic acid, and combinations thereof.

In some embodiments, core is a crystal having have at least one lateral dimension of about 100 μm to about 1000 μm. In some embodiments, the binder in the first coating layer is present in a concentration of about 0.1% to about 10% by weight of the extended release pellet. In some embodiments, the binder in the first coating layer is selected from: methylcellulose, hydroxybutylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxymethylcellulose, and combinations thereof.

In some embodiments, the binder in the second coating layer can be present in a concentration of about 0.1% to about 10% by weight of the extended release pellet. In some embodiments, the binder in the second coating layer and the drug layer are independently selected from: polyvinylpyrrolidone (e.g., povidone), copolymers of vinylpyrrolidone and vinylacetate (e.g., copovidone), hydroxypropylcellulose, hydroxyethylmethylcellulose, and combinations thereof.

In some embodiments, the dipyridamole in the therapeutic drug layer is present in a concentration of about 70% to about 95% by weight of the drug layer. The binder in the therapeutic drug layer is present in a concentration of about 0.1% to about 25% by weight of the drug layer.

In some embodiments, the enteric polymer in the extended release layer is present in a concentration of about 0.1% to about 10% by weight of the extended release pellet.

In some embodiments, the pharmaceutical capsule further comprises a sealing layer, wherein the sealing layer encompasses the second coating layer. In some embodiments, the pharmaceutical capsule releases at least 80% of the dipyridamole within 12 hours or less after oral administration to a subject. In some embodiments, the pharmaceutical capsule further comprises an immediate release aspiring composition.

The present invention is also directed to a pharmaceutical capsule comprising a dipyridamole extended release pellet comprising: (i) a core comprising an organic acid, wherein the core has an aspect ratio of 1.2 or greater; (ii) a first coating layer comprising a binder and a bulking excipient that is compatible with the organic acid, wherein the first coating layer encompasses the core forming a core-first coating layer composition having an aspect ratio of about 2 to about 1; (iii) a second coating layer comprising a binder, wherein the second coating layer encompasses the first coating layer, and wherein the second coating layer is substantially free of an organic acid; (iv) a drug layer comprising dipyridamole and a binder, wherein the drug layer encompasses the second coating layer; and (v) an extended release layer comprising an enteric polymer, wherein the extended release layer encompasses the drug layer; wherein the dipyridamole and the organic acid are not in contact.

In some embodiments, the bulking excipient is selected from: tartaric acid, citric acid, fumaric acid, succinic acid, malic acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, sodium carboxymethylcellulose, and combinations thereof.

The present invention is also directed to a process for preparing a pharmaceutical capsule, the process comprising: (A) preparing a dipyridamole extended release pellet comprising: (i) depositing a first coating layer onto a core comprising an organic acid, wherein the core has an aspect ratio of 1.2 or greater, wherein the first coating layer comprises a binder and a bulking excipient that is compatible with the organic acid in the core, forming a core-first modifying layer composition having an aspect ratio of 2 to about 1; (ii) depositing onto the first coating layer a second coating layer comprising a binder, wherein the second coating layer is substantially free from an organic acid; (iii) depositing onto the second coating layer a drug layer comprising dipyridamole and a binder; and (iv) depositing onto the drug layer an extended release layer comprising an enteric polymer, wherein the dipyridamole and the organic acid are not in contact in the pellet; (B) adding the dipyridamole extended release pellet to the pharmaceutical capsule; and (C) sealing the pharmaceutical capsule.

In some embodiments, depositing the first coating layer comprises fluid bed processing or processing using an organic solvent. In some embodiments, the process of the present invention further comprises adding an immediate release aspirin composition to the pharmaceutical capsule.

The present invention is also directed to a method of treating stroke in a subject in need thereof, the method comprising administering to the subject a pharmaceutically effective amount of a pharmaceutical capsule comprising: (i) a core comprising an organic acid, wherein the core has an aspect ratio of 1.2 or greater; (ii) a first coating layer comprising a binder and a bulking excipient that is compatible with the organic acid, wherein the first coating layer encompasses the core forming a core-first coating layer composition having an aspect ratio of about 2 to about 1; (iii) a second coating layer comprising a binder, wherein the second coating layer encompasses the first coating layer, and wherein the second coating layer is substantially free of an organic acid; (iv) a drug layer comprising dipyridamole and a binder, wherein the drug layer encompasses the second coating layer; and (v) an extended release layer comprising an enteric polymer, wherein the extended release layer encompasses the drug layer; wherein the dipyridamole and the organic acid are not in contact.

### BRIEF DESCRIPTION OF THE DRAWINGS

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 depicts a scanning electron micrograph of a core of the present invention.

FIG. 2 depicts a scanning electron micrograph of a cross-section of a core of the present invention having a first coating layer thereon.

FIG. 3 depicts a scanning electron micrograph of a cross-section of a dipyridamole extended release pellet of the present invention.

FIG. 4 depicts a schematic representation of a dipyridamole extended release pellet of the present invention.

One or more embodiments of the present invention will now be described with reference to the accompanying drawings. In the drawings, like reference numbers can indicate identical or functionally similar elements. Additionally, the left-most digit(s) of a reference number can identify the drawing in which the reference number first appears. For purposes of clarity, not every component is labeled in every figure, nor is every component of each embodiment of the invention shown where illustration is not necessary to allow those of ordinary skill in the art to understand the invention.

### DETAILED DESCRIPTION OF THE INVENTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

References to spatial descriptions (e.g., “above,” “below,” “up,” “down,” “top,” “bottom,” etc.) made herein are for purposes of description and illustration only, and should be interpreted as non-limiting upon the pellets, cores, layers, methods, and products of any method of the present invention, which can be spatially arranged in any orientation or manner.

As used herein, “about” refers to variation in the numerical value plus or minus 10% of the numerical value.

The present invention is directed to pharmaceutical capsules comprising a dipyridamole extended release pellet which comprises a core comprising a first organic acid, wherein the core has an aspect ratio of 1.2 or greater; a first coating layer comprising a second organic acid and a binder, wherein the first coating layer encompasses the core; a second coating layer comprising a binder, wherein the second coating layer encompasses the first coating layer, and wherein the second coating layer is substantially free of an organic acid; a drug layer comprising dipyridamole and a binder, wherein the drug layer encompasses the second coating layer; and an extended release layer comprising an enteric polymer, wherein the extended release layer encompasses the drug layer; wherein the dipyridamole is not in contact with the first organic acid and the second organic acid.

In some embodiments, the extended release dosage forms of the present invention are suitable for treating stroke. As used herein “stroke” is the rapidly developing loss of brain functions due to a disturbance in the blood vessels supplying blood to the brain. A stroke can be caused by ischemia (lack of blood supply) caused by thrombosis or embolism, or due to a hemorrhage. Risk factors for stroke include advanced age, hypertension (high blood pressure), previous stroke or transient ischaemic attack (TIA), diabetes, high cholesterol, cigarette smoking, atrial fibrillation, migraine with aura, and thrombophilia (a tendency to thrombosis).

As used herein, “composition” refers to equivalents thereof, including but not limited to: cores, coated cores, crystals, seeds, pellets, micro-pellets, pills, oils, gels, compressed tablets, chewable tablets, granules, spheres, lozenges, and the like. As used herein “pellet” can also refer to equivalents thereof, including but not limited to: micro-pellets, pills, oils, gels, compressed tablets, chewable tablets, granules, spheres and lozenges.

As used herein, a “pharmaceutical capsule” refers to a capsule useful for administering dipyridamole to a subject in need thereof. As used herein, “pharmaceutical” is synonymous, and used interchangeably, with “pharmaceutically acceptable,” and refers to those compounds, materials, and/or compositions which are, within the scope of sound medical judgment, suitable for contact with the tissues of human beings and animals without excessive toxicity, irritation, allergic response, or other possible complications commensurate with a reasonable benefit/risk ratio. One of skill in the art will recognize that a wide variety of pharmaceutically acceptable excipients can be used with the present invention including those listed in the Handbook of Pharmaceutical Excipients, Pharmaceutical Press 4th Ed. (2003), and Remington: The Science and Practice of Pharmacy, Lippincott Williams & Wilkins, 21st ed. (2005), which are incorporated herein by reference in their entirety.

In some embodiments, the pharmaceutical capsules can be, but are not limited to, soft or hard gelatin capsules. In some embodiments, of the invention, the pharmaceutical capsules comprise a dipyridamole extended release pellet. In some embodiments, the pharmaceutical capsules are filled with two discrete pharmaceutical compositions: an immediate release aspirin composition comprising and the dipyridamole extended release pellet. Dipyridamole and aspirin are formulated separately to decrease spoiling of dipyridamole by acetic acid (an aspirin bi-product) and to allow different release profiles for each drug. As such, aspirin is formulated to be released immediately in the stomach (at low pH) and dipyridamole is released continuously over an extended time period.

In some embodiments, the pharmaceutical capsules comprise an organic acid that increases the solubility in vivo of dipyridamole, which is a weak base with poor solubility and a pKa of 6.1. In some embodiments, the organic acid is solubilized in vivo and then protonates the dipyridamole present in the pellet, the protonated dipyridamole being more soluble and therefore having greater absorption.

As used herein, “extended release” refers to the controlled release of dipyridamole from the pharmaceutical capsule over a period of time such that for a period of about 6 hours to about 24 hours, a substantially constant systemic level of dipyridamole results in a subject administered the pharmaceutical composition. Thus, the extended release pharmaceutical compositions have a release rate that is sufficiently slow such that a subject in need thereof can be treated by administering one or more of the pharmaceutical compositions once, twice, thrice or four times within a 24 hour period. An extended release can be a delayed, a sustained, or an extended pulsatile release. In some embodiments, the pharmaceutical capsules can be administered once or twice daily.

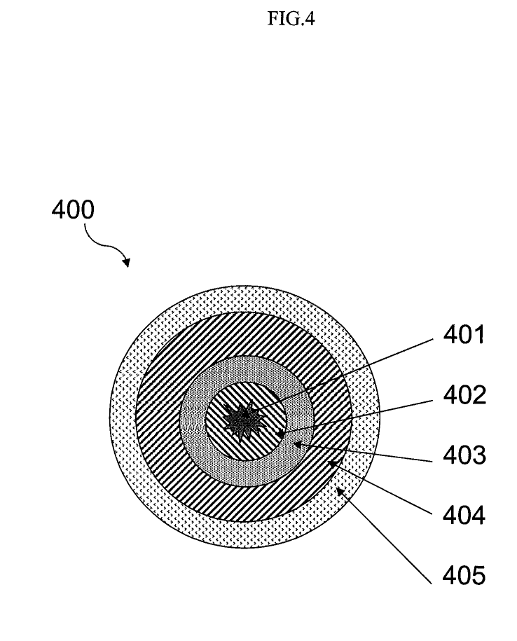

FIG. 4 provides a cross-sectional representation of a dipyridamole extended release pellet of the present invention. Referring to FIG. 4, the pellet, 400, comprises a core, 401, having first and second coating layers thereon, 402 and 403, respectively. A robust second coating layer having superior integrity prevents contact between the dipyridamole contained in the drug layer and an organic acid that is present in the core and/or the first coating layer.

The pellet further includes a drug layer, 404, and an extended release layer, 405. The drug layer, 404, comprises dipyridamole, a binder, and other optional excipients. The extended release layer, 405, comprises one or more enteric polymers. In some embodiments, the extended release layer comprises an enteric polymer that is insoluble at both low and high pH can be formulated with a second enteric polymer which is insoluble at low pH (e.g., a pH of 2) and soluble at high pH (e.g., a pH of 5 or more). Without being bound by any particular theory, a mixture of enteric polymers enables a pellet of the present invention to pass through the stomach with an intact extended release layer. When the pellet encounters a higher pH level the polymer which is insoluble at low pH and soluble at high pH will begin to be solubilized. As the extended release layer becomes permeable, fluid can enter the pellet, where it will react with and solubilize the organic acid, lowering the internal pH of the pellet and protonating the dipyridamole, thereby solubilizing the drug for absorption in the gastrointestinal tract.

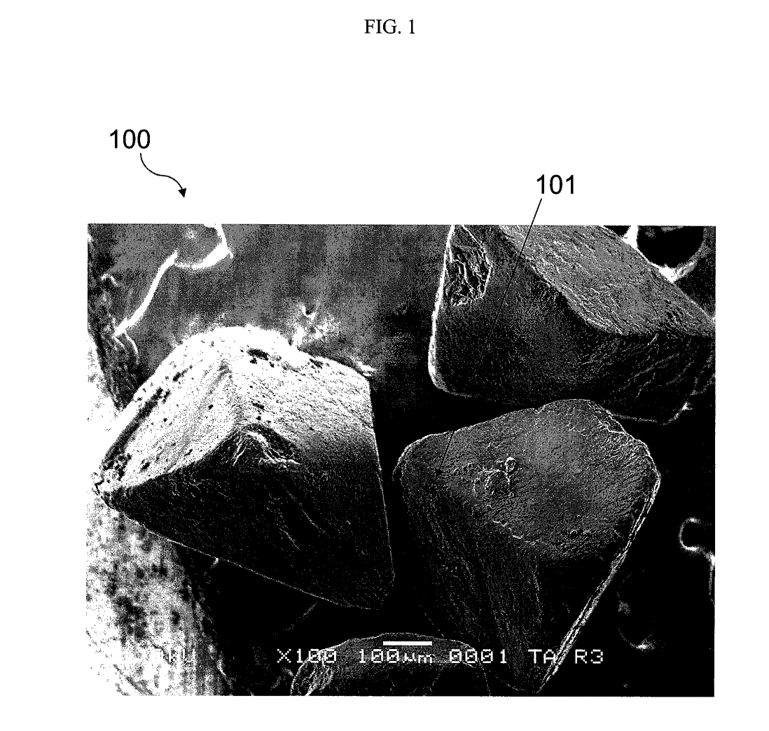

FIG. 1 provides a scanning electron micrograph image of a core of the present invention, 100. Referring to FIG. 1, in some embodiments of the present invention, a core comprises an organic acid crystal, 101.

In some embodiments, the core has at least one lateral dimension of about 100 μm to about 1000 μm, about 300 μm to about 700 μm, about 400 μm to about 800 μm, or about 400 μm to about 600 μm.

In some embodiments, the organic acid crystal is spherical. In some embodiments, the organic acid crystal is approximately spherical and in some embodiments, can be irregular. As used herein, “aspect ratio” refers to a mathematical description of a three dimensional shape of species. The aspect ratio is the longest diameter of a species, divided by the shortest diameter of the species, wherein a species can be, but is not limited to: a crystal, a pellet, a seed or a core. In some embodiments, the core has an aspect ratio of 1.2 or greater, 1.5 or greater, 2 or greater, or about 1.5 to about 3, about 2 to about 3, about 2.2 to about 3, or about 2.4 to about 3.

In some embodiments, the core comprises a crystalline acid substance, wherein the crystalline acid substance is irregularly shaped. In some embodiments, the crystalline acid substance is an organic acid crystal. Without being bound to any particular theory, the crystalline nature of the organic acid in the core enhances the absorption of dipyridamole in the gastrointestinal tract. A crystalline organic acid core will solubilize more readily and lower the internal pH of the pellet. As the internal pH level of the pellet drops, the dipyridamole is protonated, solubilizing the drug for absorption in the gastrointestinal tract.

In some embodiments, a first coating layer is applied to the irregularly shaped core to produce an approximately spherical core-first coating layer composition. In some embodiments, the core-first coating layer composition has an aspect ratio of about 1 to about 2, about 1.4 to about 1.8, or an aspect ratio of about 1 to about 1.2.

In some embodiments, the organic acid in the core is present in a concentration of about 1% to about 40%, about 5% to about 25%, or about 10% to about 15% by weight of the extended release pellet. In some embodiments, the ratio of organic acid in the core to dipyridamole is about 0.1 to about 2, about 0.2 to about 1.8, or about 0.4 to about 1.0 by weight. In some embodiments, the ratio of organic acid in the core and organic acid in the first coating layer to dipyridamole is about 0.1 to about 2, about 0.2 to about 1.8, or about 0.5 to about 1.5 by weight. In some embodiments, an organic acid present in the first coating layer is micronized, granular, or otherwise controlled in size. In some embodiments, an organic acid present in the first coating has a particle size of about 100 nm to about 100 μm, about 200 nm to about 50 μm, or about 300 nm to about 25 μm.

In some embodiments, the organic acid in the first coating layer is present in a concentration of about 10% to about 50% by weight of the extended release pellet, about 20% to about 40% by weight of the extended release pellet, or about 25% to about 35% by weight of the extended release pellet.

In some embodiments, the binder in the first coating layer is present in a concentration of about 0.1% to about 10% by weight of the extended release pellet, or about 1% to about 5%, by weight of the extended release pellet.

In some embodiments, the binder in the first coating layer is selected from: methylcellulose, hydroxybutylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxymethylcellulose, and combinations thereof. In some embodiments the binder in the first coating layer is hydroxypropylmethylcellulose.

In some embodiments, the first coating layer comprises a bulking excipient that is compatible with the organic acid. As used herein, “bulking excipient” refers a pharmaceutically acceptable excipient that adds bulk to a formulation. As used herein, “compatible” refers to those added components that are not substantially antagonistic to the organic acid. In some embodiments, the bulking excipient is selected from: tartaric acid, citric acid, fumaric acid, succinic acid, malic acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, sodium carboxymethylcellulose, and combinations thereof.

In some embodiments, the binder in the second coating layer is present in a concentration of about 0.1% to about 40%, about 0.1% to about 20%, or about 0.1% to about 10% by weight of the extended release pellet. In some embodiments, the binder in the second coating layer is selected from: polyvinylpyrrolidone (e.g., povidone), copolymers of vinylpyrrolidone and vinylacetate (e.g., copovidone), hydroxypropylcellulose, hydroxyethylmethylcellulose, and combinations thereof. In some embodiments, the second coating layer further comprises an excipient such as a glidant, an anti-adherent, a lubricant, a plasticizer or combinations thereof.

The dosage forms of the present invention provide dipyridamole having excellent stability and bioavailability. Not being bound by any particular theory, excellent bioavailability of dipyridamole using the dosage forms of the present invention is at least in part provided by the presence of an organic acid that ensures dipyridamole can be solubilized for an extended time period after oral administration, even in a weakly acidic, neutral, or basic environment. The superior stability of the dosage forms of the present invention is achieved in part by separating the organic acid from the dipyridamole.

Separation of the dipyridamole from the organic acid ensures that the organic acid cannot react with the dipyridamole during any of manufacturing, storage, shipment, and administering. In some embodiments, dipyridamole present in the drug layer is separated from an organic acid present in either of the first coating layer or the core by the second coating layer. Thus, in some embodiments the second coating layer acts as a barrier to prevent contact between dipyridamole present in the drug layer and an organic acid present in either of the first coating layer or the core. Therefore, the concentration, density, chemical composition and/or permeability of the second coating layer can ensure that the dipyridamole does not contact or react with an organic acid present in either of the core or the first coating layer.

In some embodiments, dipyridamole is present in the drug layer in a concentration of about 10% to about 55% by weight of the drug layer, or about 25% to about 45% by weight of the drug layer.

In some embodiments, the binder is present in the drug layer in a concentration of about 0.1% to about 40%, about 0.1% to about 20%, or about 1% to about 15% by weight of the drug layer. In some embodiments, the binder in the drug layer is selected from: polyvinylpyrrolidone (e.g., povidone), copolymers of vinylpyrrolidone and vinylacetate (e.g., copovidone), hydroxypropylcellulose, hydroxyethylmethylcellulose, and combinations thereof.

In some embodiments, the enteric polymer is present in the extended release layer in a concentration of about 0.1% to about 25%, about 0.1% to about 10%, or about 1% to about 5% by weight of the pellet. In some embodiments, the enteric polymer is present in the extended release layer in a concentration of about 0.1% to about 65%, about 10% to about 50%, or about 20% to about 45% by weight of the extended release layer.

In some embodiments, the enteric polymer is selected from: cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methacrylic acid copolymer, methacrylate-methacrylic acid-octyl acrylate copolymer, hydroxypropylmethylcellulose phthalate (HPMCP), polyvinylacetate phthalate (PVAP), cellulose acetylphthalate, and combinations thereof. In some further embodiments, the extended release coating layer further comprises an excipient selected from: a glidant, a lubricant, a plasticizer, and combinations thereof.

Dipyridamole is practically insoluble in water at a pH greater than 4, but is soluble in dilute acids. In some embodiments, the pH dependent solubility of dipyridamole is overcome by incorporating an organic acid into the pellets. In some embodiments, an enteric coating agent, dissolving at a pH equal to or greater than 5.5 (such as hydroxypropylmethylcellulosephthalate), is used in combination with another enteric coating polymer (such as methacrylic acid copolymer), which is soluble at a pH of about 7.0 or higher, creating an extended release coating that is increasingly more soluble as the pH of the gastrointestinal tract increases.

In some embodiments, an optional sealing layer is applied to the second coating layer, wherein the sealing layer encompasses the second coating layer. The sealing layer can comprise a polymeric carbohydrated and/or other inert pharmaceutical coating agents known to a person of ordinary skill in the art. In some embodiments, the sealing layer comprises OPADRY® II CLEAR.

In some embodiments, the extended release coating layer can be a delayed, sustained, extended or pulsative release coating.

In some embodiments, the capsule can further comprise an anti-adherent layer. In some embodiments, the capsule releases at least 80% of the dipyridamole in about 24 hours or less. In some embodiments, the capsule releases at least 80% of the dipyridamole in about 12 hours or less, and In some embodiments, the capsule releases at least 80% of the dipyridamole in about 8 hours or less after oral administration to a subject.

In some embodiments, the capsule is suitable for thrice a day oral administration, twice a day oral administration, or once a day oral administration. In some embodiments, the pharmaceutical capsule further contains an immediate release aspirin composition. As used herein, “immediate release” refers to a composition that begins to release an active agent immediately upon administration to a subject. In some embodiments, the pharmaceutical capsule comprises an immediate release aspirin composition comprising about 5 mg to about 50 mg of aspirin. The immediate release aspirin composition can be present as, e.g., a tablet, pellets, micro-pellets, pills, oils, gels, compressed tablets, chewable tablets, granules, spheres, lozenges, and the like, and combinations thereof.

The present invention is directed to a process for preparing a pharmaceutical capsule comprising: (A) preparing a dipyridamole extended release pellet comprising: (i) depositing a first coating layer onto a core comprising an organic acid, wherein the core has an aspect ratio of about 2 or greater, the first coating layer comprising a binder and a bulking excipient that is compatible with the organic acid in the core; (ii) depositing onto the first coating layer a second coating layer comprising a binder, wherein the second coating layer is substantially free from an organic acid; (iii) depositing onto the second coating layer a drug layer comprising dipyridamole and a binder; and (iv) depositing onto the drug layer an extended release layer comprising an enteric polymer, wherein the dipyridamole and the organic acid are not in contact in the pellet; (B) adding the dipyridamole extended release pellet to the pharmaceutical capsule; and (C) sealing the pharmaceutical capsule. In some embodiments, the process further comprises adding an immediate release aspirin composition to the pharmaceutical capsule. In some embodiments, depositing the first coating layer on the core comprises fluid bed processing or processing using an organic solvent.

In some embodiments, due to the high aqueous solubility of an organic acid present in the core, the first coating layer is applied using a non-aqueous organic solvent, such as ethanol, acetone and isopropyl-alcohol in the first coating dispersion/solvent system. In some embodiments, the first coating layer can be achieved by powder layering, and solution or dispersion coating in a fluid-bed or high shear mixer/granulator.

Referring to FIG. 4 a first coating layer, 402, is applied to irregularly shaped organic acid crystals, 401, to create a regularly shaped surface onto which the second coating layer, 403, is deposited. The second coating layer, 403, is deposited onto the first coating layer, 402. Without being bound by any particular theory, the smoothness (or roughness) of the first coating layer, 402, can enhance solvent evaporation from the surface and decreases the amount of surface residual solvent, in doing so reducing the amount of solvent available for interaction with the organic acid and subsequent spoilage of dipyridamole. The second coating layer, 403, can thus be efficiently applied to the first coating layer to provide an integral coating having a weight gain of about 20% or more.

The present invention is also directed to a method of treating stroke in a subject in need thereof, the method comprising administering to the subject a pharmaceutically effective amount of a pharmaceutical capsule comprising: a core comprising an organic acid, wherein the core has an aspect ratio of 2 or greater; a first coating layer comprising a binder and a bulking excipient that is compatible with the organic acid, wherein the first coating layer encompasses the core and the core-first coating layer composition has an aspect ratio of about 2 to about 1; a second coating layer comprising a binder, wherein the second coating layer encompasses the first coating layer, and wherein the second coating layer is substantially free of an organic acid; a drug layer comprising dipyridamole and a binder, wherein the drug layer encompasses the second coating layer; and an extended release layer comprising an enteric polymer, wherein the extended release layer encompasses the drug layer; wherein the dipyridamole and the organic acid are not in contact. In some embodiments, the method further comprises a pharmaceutical capsule comprising an immediate release aspirin composition.

As used herein, “treating stroke” and “treatment of stroke” refer to the reduction of risk of stroke in patients who have had transient ischemia of the brain or completed ischemic stroke due to thrombosis; the alleviation of symptoms associated with stroke; diminishment of the extent of the condition associated with stroke; stabilization (i.e., not worsening) of the state of the condition or disorder associated with stroke; delay in onset or slowing of the condition or disorder progression associated with stroke; amelioration of the condition or disorder state associated with stroke; remission (whether partial or total) of the condition, disorder or disease associated with stroke, whether detectable or undetectable; or enhancement or improvement of the condition, disorder or disease associated with stroke. Treatment includes eliciting a clinically significant response, without excessive levels of side effects. Treatment also includes prolonging survival as compared to expected survival if not receiving treatment.

As used herein, “pharmaceutically effective amount” refers to an amount effective, at dosages and for periods of time necessary, to achieve the desired therapeutic result. In some embodiments the pharmaceutically effective amount refers to two or more pharmaceutical capsules of the invention together totaling a pharmaceutically effective amount, or one pharmaceutical capsule of the invention containing the pharmaceutically effective amount. In some embodiments, one or more pharmaceutical capsules are administered thrice a day, twice a day, or once a day to a subject in need thereof.

As used herein, “a subject in need thereof” refers to a subject that is experiencing a particular condition associated with stroke, as observed or identified by a physician, physician's assistant, nurse, or other healthcare professional. It is not intended that the present invention be limited to any particular signs or symptoms associated with stroke. Thus, it is intended that the present invention encompass subjects that are experiencing any range of conditions, wherein the subject exhibits at least one or more indicia associated with stroke.

In some embodiments, the present invention is directed to a method of treating stroke wherein the capsule releases at least 80% of the dipyridamole in about 12 hours or less, or about 8 hours or less after oral administration to a subject.

The following examples are for the purpose of illustration of the invention only and are not intended in any way to limit the scope of the present invention. It will thus be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

### EXAMPLES

### Example 1A

### Preparation of a First Coating Layer

METHOCEL® K100 and tartaric acid were added slowly to a vortex of ethanol and gently mixed in a suitable container equipped with an air-operated mixer, until a lump-free dispersion was obtained. Water was then added and mixed, producing a first coating dispersion of 25% tartaric acid/METHOCEL® K100 in ethanol and water.

### Example 1B

### Preparation of a Second Coating Layer

Povidone was added slowly to a vortex of isopropyl alcohol and gently mixed in a suitable container equipped with an air-operated mixer, until a clear dispersion was obtained. Talc was then added slowly and mixed until a lump-free dispersion was obtained, producing a second coating dispersion of 20% povidone/copovidone and talc in ethanol and water.

### Example 1C

### Preparation of a OPADRY® II Clear Sealing Layer

OPADRY® II Clear was added to a vortex of water in a suitable container equipped with an air-operated mixer, and gently mixed until a clear dispersion was obtained, producing a 5% OPADRY® II seal coat.

### Example 1D

### Preparation of Drug Layer

Simethicone and povidone were added slowly to a vortex of water and gently mixed in a suitable container equipped with an air-operated mixer, until a clear dispersion was obtained. Dipyridamole was then added slowly and mixed until a lump-free dispersion was obtained, producing a therapeutic drug coating dispersion of 18% dipyridamole.

### Example 1E

### Preparation of an Extended Release Layer

EUDRAGIT® S100 and EUDRAGIT® HP-55 were added slowly to a vortex of ethyl alcohol and gently mixed in a suitable container equipped with an air-operated mixer, until a lump-free dispersion was obtained. Water was then added slowly until a clear solution was obtained, and triacetin and talc were then added to produce a 5% extended release coating dispersion.

### Example 2

### Preparation of a Dipyridamole Extended Release Pellet

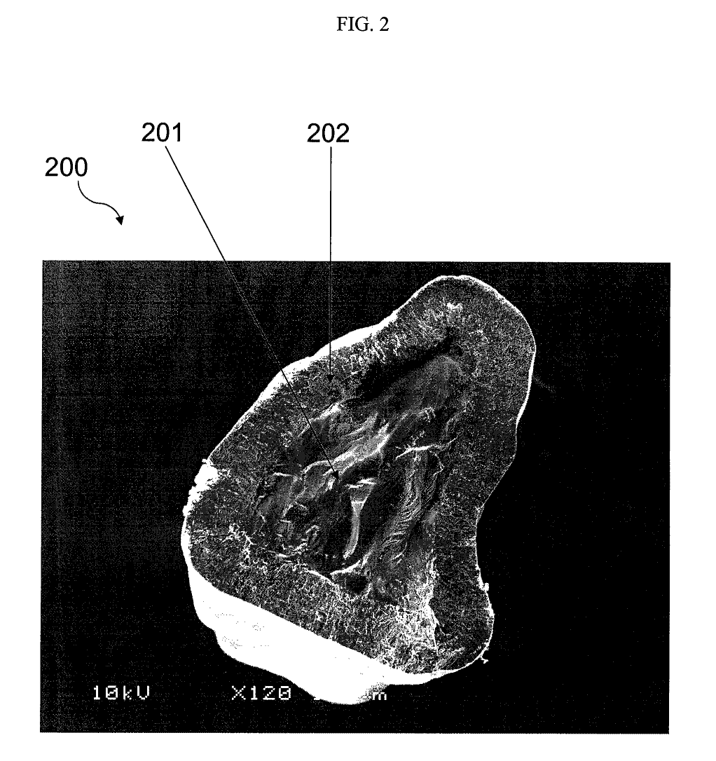

A first coating layer (Example 1A) was applied to core (e.g., tartaric acid crystals) by dispersion coating on a fluid-bed at about 35° C. to about 38° C., and screened through U.S. standard sieve #40, and #20 to produce a core having a first coating layer thereon. FIG. 2 provides an electron micrograph of a cross-section of the tartaric acid cores having a first coating layer thereon. Referring to FIG. 2, the electron micrograph, 200, shows the core, 201, having a first coating layer, 202, thereon.

A second coating layer (Example 1B) was applied to the first coated layer by dispersion coating on a fluid-bed at about 30° C. to about 38° C., and screened through U.S. Standard Sieve #40, and #16 to produce a tartaric acid core with a second coating layer.

An OPADRY® II Clear sealing layer (Example 1C) was applied to the second coated layer by dispersion coating on a fluid-bed at about 34° C. to about 38° C., and screened through U.S. Standard Sieve #40, and #16 to produce an OPADRY® II Clear sealing layer.

A drug layer (Example 1D) was applied to the OPADRY® II Clear sealing layer by dispersion coating on a fluid-bed at about 34° C. to about 38° C., and screened through U.S. Standard Sieve #40, and #16 to produce a drug coating layer.

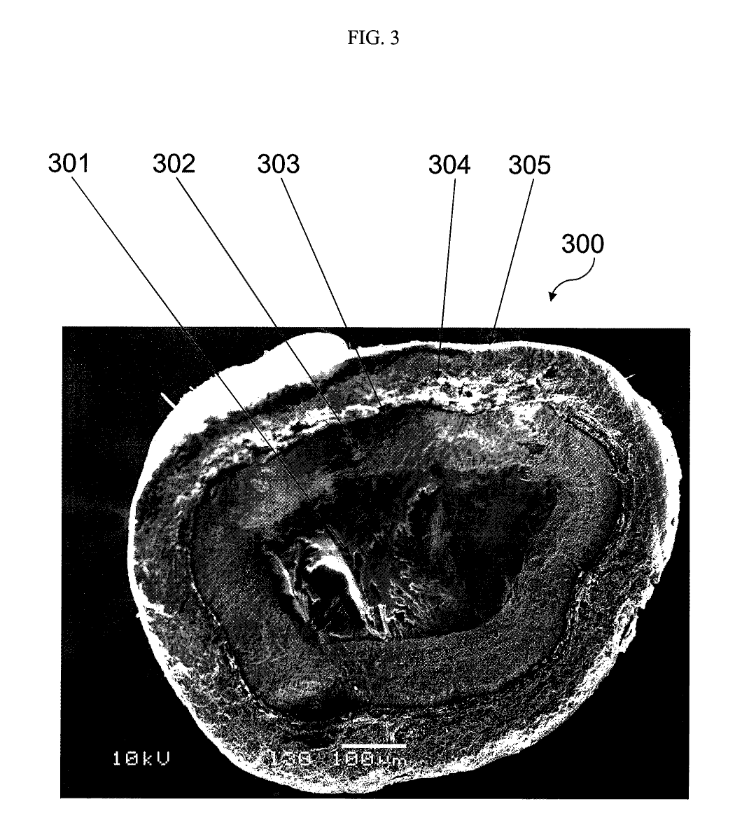

An extended release layer (Example 1E) was applied to the drug coating layer by dispersion coating on a fluid-bed at about 30° C. to about 34° C., and screened through U.S. Standard Sieve #40, and #16 to produce the extended release coating and the completed dipyridamole extended release pellet. FIG. 3 provides an electron micrograph of a cross-section of a dipyridamole extended release pellet. Referring to FIG. 3, the electron micrograph, 300, shows the core, 301, having a first coating layer, 302, a second coating layer, 303, a drug layer, 304, and an extended release layer, 305, thereon. For specific gram quantities per dose see Table 1.

TABLE 1
Formulation composition for dipyridamole extended release
pellets as prepared by Examples 1A-1E and Example 2.

| # | Ingredient | mg/dose |
|---|---|---|
| | Core | |
| 1 | Tartaric Acid, NF | 80 |
| | (irregularly shaped crystals, retained | |
| | on #40 Mesh) | |
| | First Coating Layer | |
| 2 | Tartaric Acid (Extra Fine Granular) | 180 |
| 3 | Hypromellose 2208, USP (100 cPs) (METHOCEL ® | 7 |
| | K100-LV Premium) | |
| 4 | Alcohol, USP (Ethyl Alcohol 190 Proof)* | |
| 5 | Purified Water, USP* | |
| | Second Coating Layer | |
| 6 | Povidone, USP (Plasdone K-25) | 21 |
| 7 | Copovidone, NF (Kollidon VA64) | 21 |
| 8 | Talc, USP | 11 |
| 9 | Alcohol, USP (Ethyl Alcohol 190 Proof)* | |
| 10 | Purified Water, USP* | |
| | Sealing Layer | |
| 11 | OPADRY ® II Clear Y-19-7483 | 10 |
| 12 | Purified Water, USP | |
| | Drug Layer | |
| 13 | Dipyridamole, USP (Micronized) | 200 |
| 14 | Povidone, USP (KOLLIDON 90F or equivalent) | 59 |
| 15 | Simethicone, USP (Medical Antifoam A Compound) | 1 |
| 16 | Purified Water, USP* | |
| | Extended Release Layer | |
| 17 | Methacrylic Acid Copolymer, NF (EUDRAGIT ® S100) | 15 |
| 18 | Hypromellose Phthlate, NF (HPMCP, EUDRAGIT ® | 5 |
| | HP-55) | |
| 19 | Talc, USP | 8 |
| 20 | Triacetin, USP | 5 |
| 21 | Alcohol, USP (Ethyl Alcohol 190 Proof) | |
| 22 | Purified Water, USP | |
| | Anti-Adherent | |
| 23 | Talc, USP | 3 |
| | Total | 626 |

Tartaric acid solubility in H₂0 is 1.33 g/mL, dipyridamole pKₐ 6.1
*Alcohol and water are not present in the final pellets.

### CONCLUSION

These Examples illustrate possible embodiments of the present invention. While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

It is to be appreciated that the Detailed Description section, and not the Summary and Abstract sections, is intended to be used to interpret the claims. The Summary and Abstract sections can set forth one or more, but not all exemplary embodiments of the present invention as contemplated by the inventor(s), and thus, are not intended to limit the present invention and the appended claims in any way.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A pharmaceutical capsule comprising:
a dipyridamole extended release pellet comprising:
(i) a core comprising a first organic acid, wherein the core has an aspect ratio of 1.2 or greater;
(ii) a first coating layer comprising a second organic acid and a binder, wherein the first coating layer encompasses the core;
(iii) a second coating layer comprising a binder, wherein the second coating layer encompasses the first coating layer, and wherein the second coating layer is substantially free of an organic acid;
(iv) a drug layer comprising dipyridamole and a binder, wherein the drug layer encompasses the second coating layer; and
(v) an extended release layer comprising an enteric polymer, wherein the extended release layer encompasses the drug layer;
wherein the dipyridamole is not in contact with the first organic acid and the second organic acid.

2. The pharmaceutical capsule of claim 1, wherein a composition of the core and the first coating layer encompassing the core has an aspect ratio of about 2 to about 1.

3. The pharmaceutical capsule of claim 1, wherein a ratio of the first organic acid and second organic acid to the dipyridamole is about 0.1 to about 2 by weight.

4. The pharmaceutical capsule of claim 1, wherein the first organic acid and the second organic acid are independently selected from: tartaric acid, citric acid, fumaric acid, succinic acid, malic acid, and combinations thereof.

5. The pharmaceutical capsule of claim 1, wherein the core is a crystal having at least one lateral dimension of about 100 μm to about 1000 μm.

6. The pharmaceutical capsule of claim 1, wherein the binder in the first coating layer is present in a concentration of about 0.1% to about 10% by weight of the extended release pellet.

7. The pharmaceutical capsule of claim 1, wherein the binder in the first coating layer is selected from: methylcellulose, hydroxybutylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxymethylcellulose, and combinations thereof.

8. The pharmaceutical capsule of claim 1, wherein the binder in the second coating layer is present in a concentration of about 0.1% to about 10% by weight of the extended release pellet.

9. The pharmaceutical capsule of claim 1, wherein the binder in the second coating layer and the drug layer are independently selected from: polyvinylpyrrolidone, copolymers of vinylpyrrolidone and vinylacetate, hydroxypropylcellulose, hydroxyethylmethylcellulose, and combinations thereof.

10. The pharmaceutical capsule of claim 1, wherein the dipyridamole in the drug layer is present in a concentration of about 70% to about 95% by weight of the drug layer.

11. The pharmaceutical capsule of claim 1, wherein the binder in the drug layer is present in a concentration of about 0.1% to about 25% by weight of the drug layer.

12. The pharmaceutical capsule of claim 1, wherein the enteric polymer in the extended release layer is present in a concentration of about 0.1% to about 10% by weight of the extended release pellet.

13. The pharmaceutical capsule of claim 1, wherein the pharmaceutical capsule further comprises a sealing layer, wherein the sealing layer encompasses the second coating layer.

14. The pharmaceutical capsule of claim 1, wherein the capsule releases at least 80% of the dipyridamole within about 12 hours or less after oral administration to a subject.

15. The pharmaceutical capsule of claim 1, wherein the pharmaceutical capsule further comprises an immediate release aspirin composition.

16. A pharmaceutical capsule comprising:
a dipyridamole extended release pellet comprising:
(i) a core comprising an organic acid, wherein the core has an aspect ratio of 1.2 or greater;
(ii) a first coating layer comprising a binder and a bulking excipient that is compatible with the organic acid, wherein the first coating layer encompasses the core, forming a core-first coating layer composition having an aspect ratio of about 2 to about 1;
(iii) a second coating layer comprising a binder, wherein the second coating layer encompasses the first coating layer, and wherein the second coating layer is substantially free of an organic acid;
(iv) a drug layer comprising dipyridamole and a binder, wherein the drug layer encompasses the second coating layer; and
(v) an extended release layer comprising an enteric polymer, wherein the extended release layer encompasses the drug layer;
wherein the dipyridamole and the organic acid are not in contact.

17. The pharmaceutical capsule of claim 16, wherein a ratio of the organic acid to the dipyridamole is about 0.1 to about 2 by weight.

18. The pharmaceutical capsule of claim 16, wherein the organic acid is selected from: tartaric acid, citric acid, fumaric acid, succinic acid, malic acid, and combinations thereof.

19. The pharmaceutical capsule of claim 16, wherein the core is a crystal having at least one lateral dimension of about 100 μm to about 1000 μm.

20. The pharmaceutical capsule of claim 16, wherein the bulking excipient is selected from: tartaric acid, citric acid, fumaric acid, succinic acid, malic acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, sodium carboxymethylcellulose, and combinations thereof.

21. The pharmaceutical capsule of claim 16, wherein the dipyridamole in the drug layer is present in a concentration of about 70% to about 95% by weight of the drug layer.

22. The pharmaceutical capsule of claim 16, wherein the binder in the drug layer is present in a concentration of about 0.1% to about 25% by weight of the drug layer.

23. The pharmaceutical capsule of claim 16, wherein the capsule releases at least 80% of the dipyridamole within about 12 hours or less after oral administration to a subject.

24. The pharmaceutical capsule of claim 16, wherein the pharmaceutical capsule further comprises an immediate release aspirin composition.

25. A process for preparing a pharmaceutical capsule, the process comprising:
(A) preparing a dipyridamole extended release pellet comprising:
(i) depositing a first coating layer onto a core comprising an organic acid, wherein the core has an aspect ratio of 1.2 or greater, wherein the first coating layer comprises a binder and a bulking excipient that is compatible with the organic acid in the core, forming a core-first coating layer composition having an aspect ratio of about 2 to about 1;
(ii) depositing onto the first coating layer a second coating layer comprising a binder, wherein the second coating layer is substantially free from an organic acid;
(iii) depositing onto the second coating layer a drug layer comprising dipyridamole and a binder; and
(iv) depositing onto the drug layer an extended release layer comprising an enteric polymer,
wherein the dipyridamole and the organic acid are not in contact in the pellet;
(B) adding the dipyridamole extended release pellet to the pharmaceutical capsule; and
(C) sealing the pharmaceutical capsule.

26. A pharmaceutical capsule prepared by the process of claim 25.

27. The process of claim 25, wherein depositing the first coating layer comprises fluid bed processing or processing using an organic solvent.

28. The process of claim 25, further comprising adding an immediate release aspirin composition to the pharmaceutical capsule.

29. A method of treating stroke in a subject in need thereof, the method comprising administering to the subject a pharmaceutically effective amount of a pharmaceutical capsule comprising a dipyridamole extended release pellet comprising:
(i) a core comprising an organic acid, wherein the core has an aspect ratio of 1.2 or greater;
(ii) a first coating layer comprising a binder and a bulking excipient that is compatible with the organic acid, wherein the first coating layer encompasses the core, forming a core-first coating layer composition having an aspect ratio of about 2 to about 1;
(iii) a second coating layer comprising a binder, wherein the second coating layer encompasses the first coating layer, and wherein the second coating layer is substantially free of an organic acid;
(iv) a drug layer comprising dipyridamole and a binder, wherein the drug layer encompasses the second coating layer; and
(v) an extended release layer comprising an enteric polymer, wherein the extended release layer encompasses the drug layer;
wherein the dipyridamole and the organic acid are not in contact.

30. The method of claim 29, wherein the pharmaceutical capsule further comprises an immediate release aspirin composition.
